(19) United States
(12) Patent Application Publication
(10) Pub. No.: US 2006/0116509 A1
(43) Pub. Date: Jun. 1, 2006

(54) Manufacture of limonoid compounds

(75) Inventors: Gary D. Manners, El Sobrante, CA (US); Andrew P. Breksa III, Richmond, CA (US)

Correspondence Address: USDA-ARS-OFFICE OF TECHNOLOGY TRANSFER;PATENT ADVISORS OFFICE, WESTERN REGIONAL RESEARCH CENTER, 800 BUCHANAN ST, ALBANY, CA, 94710, US

(73) Assignee: The United States of America, as represented by the Secretary of Agriculture, Washington, DC (US)

(21) Appl. No.: 11/289,240

(22) Filed: Nov. 29, 2005

Related U.S. Application Data
(60) Provisional application No. 60/631,796, filed on Nov. 29, 2004.

(51) Int. Cl. C07H 1/06; C07H 1/08

(52) U.S. Cl. 536/127

(57) ABSTRACT

One embodiment of the present invention is directed to methods for obtaining limonoid A-ring lactone acid salts, limonoid glycoside monocarboxylic acids, limonoid glycoside dicarboxylic acids, limonoid glycoside monocarboxylic acid salts, and limonoid glycoside dicarboxylic acid salts from Citrus fruit, fruit parts or extracts, and processing by-products. In one embodiments the methods are practiced on a production scale. In another embodiment the invention is directed to products obtained by the methods. In still another embodiment the invention provides pharmaceutical and nutraceutical compositions comprising isolated pure limonoid compounds.

### CROSS REFERENCES TO RELATED APPLICATIONS

This application claims priority to U.S. Provisional Patent Application No. 60/631,796, filed Nov. 29, 2004 which is herein incorporated by reference in its entirety.

### FIELD OF THE INVENTION

The present invention relates to methods for manufacturing limonoid compounds. The invention provides methods for isolating limonoid A-ring lactone acid salts, limonoid glycoside monocarboxylic acids, limonoid glycoside dicarboxylic acids, limonoid glycoside monocarboxylic acid salts, and limonoid glycoside dicarboxylic acid salts. The invention is also directed to products obtained by the methods.

### BACKGROUND OF THE INVENTION

Limonoid compounds are a group of chemically related triterpenoid compounds found in the Rutaceae and Meliaceae plant families. Certain of the limonoid compounds are among the bitter principals found in citrus fruits. The bitterness that can develop in citrus juices due to limonoids is of significant negative economic impact. Therefore, limonoid compounds are of major interest to the citrus industry (see e.g., Hasegawa et al. (1999) In Flavor Chemistry: 30 Years of Progress, Teranishi, R.; Wick, E. L.; Hornstein, I. eds., Kluwer/Plenum Publishers: New York; 89-106 and U.S. Pat. No. 5,817,354).

Although interest in limonoid compounds first arose out of the citrus industry's search for the source of delayed bitterness in citrus juices, limonoid compounds have since been shown to have beneficial effects on human health. For example, U.S. patent application Publication No. 20010055627, discloses the use of limonoid compounds for the treatment of hypercholesterolemia, hyperlipidemia and atherosclerosis. Limonoids have also been found to have anti-carcinogenic activity (see e.g., U.S. Pat. No. 6,239,114 and U.S. Pat. No. 6,251,400 and Guthrie, N., et al. (2000) In: Citrus Limonoids: Functional Chemicals in Agriculture and Foods, Berhow, M., Hasegawa, S. and Manners, G. eds. ACS Symposium series 758. pp. 164-174). Without being bound by theory, it is thought by some that at least one way in which limonoid compounds detoxify chemical carcinogens is by inducing the liver glutathione —S— transferase enzyme system (see e.g., U.S. Pat. No. 5,041,425 and Lam et al., 1994, Food Technol. 48:104-108), but other mechanisms known and still unknown may also play a role.

Methods have been developed that reduce or eliminate the concentration of may be debittered by removal of limonin using cellulose esters, such as cellulose acetate and/or cellulose acetate butyrate, as adsorbents. The juices can be treated either before or after packaging into containers (see e.g., U.S. Pat. No. 3,989,854 and U.S. Pat. No. 6,169,044).

However, as noted above limonoid compounds also have beneficial health effects, and as a result, there is now interest in isolating limonoid compounds for the manufacture of functional food additives in nutritional supplements, nutraceuticals, pharmaceuticals and the like, and for use in therapeutic, nutritional and clinical settings (see e.g., Mazza, G. (1998) Functional Foods, CRC Press).

Accordingly, procedures for isolating limonoid compounds have been developed. For example, procedures for the extraction and isolation of both aglycones and glucosides have been established to obtain concentrated sources of various limonoids (Lam, L. K. T. et al., 1994, in Food Phytochemicals for Cancer Prevention, eds. M. Huang, T. Osawa, C. Ho and R. T. Rosen, ACS Symposium Series 546, p 209).

Some of the isolation methods available in the art are also useful at production scale. For example, Miyake et al. ((1999) In Citrus limonoids. Functional chemicals in agriculture and foods, Berhow, M A, Hasegawa, S. Manners, G. D., eds., American Chemical Society: Washington, D.C. pgs 97-106) disclose a method for carbon dioxide extraction of limonoid aglycones and the purification of limonoid glucosides. The method may be used for simple debittering or for isolation of aglycone mixtures. The method disclosed in U.S. Pat. No. 5,734,046 recites a method for the production of limonoid glucoside mixtures. Unfortunately, these methods are limited to the isolation of complex mixtures of limonoid glucosides and limonoid aglycones. Thus, such methods do not provide for the isolation on a large scale of individual species of limonoid compound.

Purified individual limonoid aglycones have been obtained utilizing a solvent extraction and enzyme conversion method (Herman Z., et al. (1992) In: Modern Methods of Plant Analysis New Series, Volume 14 Seed Analysis, Linskens, H. F. and Jackson, J. F. eds. Springer-Verlag Berlin, pgs 361-375, which is incorporated herein by reference in its entirety), and pure limonoid glucosides have been isolated from limonoid glucoside mixtures (Bennett R. D., et al. (1991) Phytochemistry 30:3803-3805; Braddock R. J. et al.( 2001) J. Agric. Food Chem. 49:5982-5988; Miyake M. et al. (1992a) Phytochemistry 31:1044-1046; Miyake M. et al. (1992b) Phytochemistry (Life Sci. Adv.) 11:51-53). Unfortunately however, the disclosed methods are not practical for production on a large scale. Therefore, for the development of beneficial health products using individual limonoid compounds as ingredients, none of the isolation procedures disclosed prior to the invention are satisfactory, since none achieve single pure limonoid aglycones or limonoid glucosides on a production scale.

Thus, there exists a need to produce individual limonoid compound in pure form, on a production scale. As will be clear from the following disclosure, the present invention provides for this and other needs.

### SUMMARY OF THE INVENTION

In one aspect, the present invention provides a method for separating limonoid glycoside mono-carboxylic acids from limonoid glycoside di-carboxylic acids in an aqueous solution. In one embodiment, the method comprises acidifying the aqueous solution to achieve a pH of less than 6.5; applying the acidified aqueous solution to a strong anion ion exchange resin; eluting the strong anion ion exchange resin with a linear gradient of an ionic solvent; and collecting eluate fractions that comprise separated limonoid glycoside mono-carboxylic acids and separated limonoid glycoside di-carboxylic acid.

In another aspect, the invention provides method steps for separating an individual species of limonoid glycoside mono-carboxylic acid from other species of limonoid glycoside mono-carboxylic acid in an aqueous solution. In one embodiment the steps comprise applying the collected eluate fractions comprising limonoid glycoside mono-carboxylic acids to an aromatic-selective chromatographic medium; eluting the aromatic-selective chromatographic medium with a polar solvent; and collecting eluate fractions comprising the separated individual species of limonoid glycoside mono-carboxylic acid.

In another aspect, the method provides steps for separating an individual species of limonoid glycoside di-carboxylic acid from other species of limonoid glycoside di-carboxylic acids in an aqueous solution. In an exemplary embodiment the steps comprise applying the collected eluate fractions comprising limonoid glycoside di-carboxylic acids to an aromatic-selective chromatographic medium; eluting the aromatic-selective chromatographic medium with a polar solvent; and collecting eluate fractions comprising the separated individual species of limonoid glycoside di-carboxylic acid. In one exemplary embodiment, the aromatic-selective chromatographic medium is a phenyl-bonded silica medium.

In another aspect the invention provides a method for producing a pure crystalline form of an individual species of limonoid glycoside carboxylic acids. In an exemplary embodiment the method comprises the steps of combining eluate fractions comprising the same isolated individual species of limonoid glycoside carboxylic acid; applying the combined eluate fractions to a styrene divinylbenzene resin; eluting the styrene divinylbenzene resin with a polar solvent; collecting eluate fractions comprising the individual species of limonoid glycoside carboxylic acid; evaporating the polar solvent present in the collected fractions to produce a limonoid glycoside carboxylic acid concentrate; and fractionally crystallizing the limonoid glycoside carboxylic acid concentrate,

In another aspect the invention provides a method for producing an amorphous solid comprising the alkali metal salt of the individual species of a limonoid glycoside mono- or di-carboxylic acid. In an exemplary embodiment, the method comprises the steps of combining eluate fractions comprising the same individual species of limonoid glycoside mono- or di-carboxylic acid; adding an aqueous alkali metal base to the combined fractions to make the combined fractions alkaline; applying the alkaline eluate fractions to a styrene divinylbenzene resin; washing the styrene divinylbenzene resin with water to remove excess alkali; eluting the styrene divinylbenzene resin with a polar solvent; collecting eluate fractions comprising the individual species of limonoid glycoside mono- or di-carboxylic acid; and evaporating the polar solvent present in the collected. In one embodiment, the aqueous alkali metal base is aqueous NaOH.

In another aspect the invention provides an alkali metal salt of an isolated limonoid glycoside carboxylic acid made according to the methods disclosed herein.

In another aspect the invention provides a method of isolating limonoid aglycones in the form of limonoid A ring lactone limonates, from a composition comprising seed solids. In an exemplary embodiment, the method comprises suspending the composition comprising seed solids in a solution of aqueous alkali metal base to form a seed solid suspension; homogenizing the seed solid suspension to form a homogenate; storing the homogenate for a time period of at least 10 hours to form limonoid A-ring lactone limonates from limonoid aglycones; processing the homogenate to form a solid phase and an alkaline aqueous phase; collecting the alkaline aqueous phase; applying the alkaline aqueous phase onto a phenyl bonded silica gel; eluting the phenyl-bonded silica gel with a polar solvent; and collecting eluate fractions comprising limonoid A-ring lactone limonates.

In an exemplary embodiment, the method further comprises combining collected fractions comprising limonoid A-ring lactone limonates; loading the collected fractions onto a styrene divinylbenzene resin; eluting the styrene divinylbenzene resin with a polar solvent; collecting eluate fractions comprising limonoid A-ring lactones; and evaporating the polar solvent from the collected fractions to yield an amorphous solid comprising the alkali metal salts of limonoid A-ring lactone limonates.

In another embodiment, the invention provides pharmaceutical and nutraceutical compositions comprising isolated pure limonoid compounds.

Other features, objects and advantages of the invention will be apparent from the detailed description which follows.

### BRIEF DESCRIPTION OF THE DRAWINGS

FIG. 1 shows an exemplary isolation and purification scheme for the isolation and purification of limonoid glycosides, limonoid glycoside alkali metal salts, and limonoate A-ring lactone alkali metal salts from citrus seeds.

FIG. 2 shows an exemplary isolation and purification scheme for the isolation and purification of individual limonoid glycosides and limonoid glycoside alkali metal salts of limonin glycosides from citrus sources.

FIG. 3 shows the chemical/biochemical interconversion of limonoid A-ring lactones to limonoid glycosides and limonoid aglycones.

FIG. 4 shows structures of some exemplary citrus limonoids.

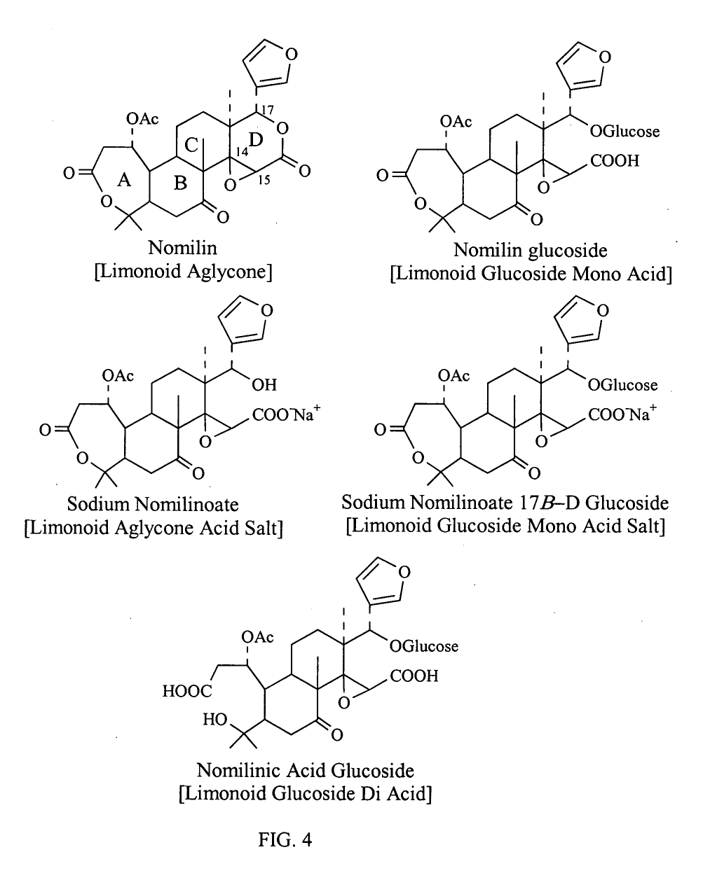

### DETAILED DESCRIPTION OF THE INVENTION

Definitions

As used herein, the term “limonoid compound” refers to any limonoid based chemical compound. The term “limonoid compounds” refers inclusively to limonoid aglycones, limonoate A-ring lactones, limonoid glycosides including, but not limited to limonoid glucosides, limonoid carboxylic acids including, but not limited to limoinoid glycoside mono- and di-carboxylic acids.

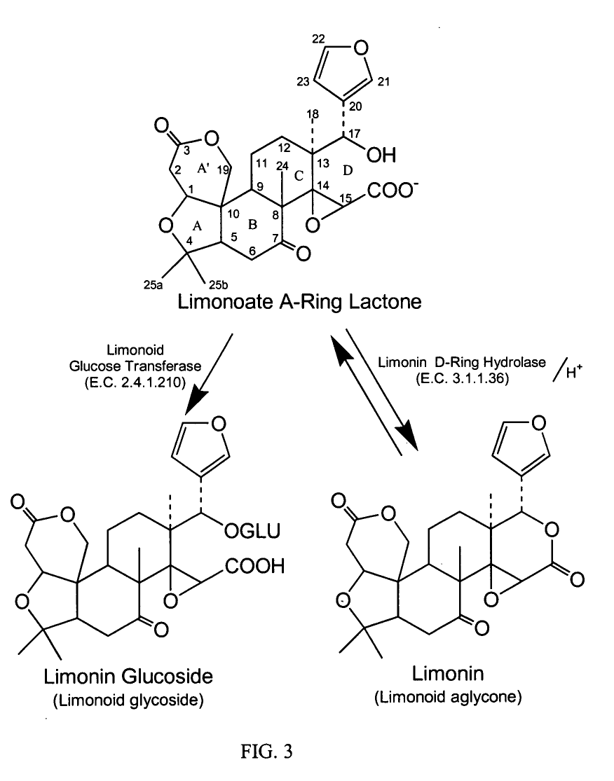

The term “limonoid aglycone” refers to triterpenoid compounds that display a furan ring attached at C-17, two lactone rings on opposite sides of the molecule e.g., limonin, nomilin, obacunone, and deacetylnomilin. Often a “limonoid aglycone” will also display a C14,15 epoxide and/or an oxygenated C-7. In some embodiments, the lactone ring on the right side of the molecule is associated with an oxygen at C-17. There are several limonoids that have fragmented lactone rings on the right side of the molecule. Thus in some embodiments, a limonoid aglycone” refers to a highly oxygenated triterpenoid compound that displays a furan ring attached at C-17, a lactone ring on the right side of the molecule incorporating an alcholic oxygen at C-17 and a lactone ring or fragmented lactone ring on the left side of the molecule. In another embodiment, this form of limonoid aglycone also displays an oxygen functionality at C-7 and a C14, 15 epoxide. Limonin (FIG. 3) is an exemplary limonoid aglycone. Limonin is a water insoluble limonoid aglycone. Limonin is a major component among limonoid aglycones present in citrus seeds, and is a major contributor to bitterness in citrus.

Limonin is generated from “limonoate A-ring lactone” (see e.g., FIG. 3) in the juice of citrus subjected to physical stress or freezing through the action of acid and the endogenous enzyme, limonin D-ring lactone hydrolase (FIG. 3) (Breksa III, A. P., Manners, G. D. (2004) J. Agric. Food Chem. 52:3772-3775 which is incorporated herein by reference). “Limonoate A-ring lactone” or “limonoid limonate” is not a limonoid aglycone but rather a hydrolyzed lactone ring derivative of a limonoid aglycone.

Unlike limonin, “limonoate A-ring lactone” is water-soluble at neutral or basic pH. “Limonoate A-ring lactone” is tasteless. During the process of fruit maturation, limonoate A-ring lactone is converted to limonoid glycoside. Limonoate A-ring lactones are water soluble at neutral or basic pH and are tasteless.

The term “limonoid glycoside” refers to limonoid compounds that are carboxylic acid limonoids derived from hydrolyzed lactones, wherein the alcoholic oxygen of the hydrolyzed lactone ring is glycosolated. Thus, limonoid glycosides comprise derivatives of A-ring and D-ring δ-hydroxy carboxylic acid forms of citrus limonoids. In general, “limonoid glycosides” are limonoid A-ring lactones that contain one or more sugar moieties attached via a δ-glycosidic linkage at C-17. An exemplary limonoid glycoside is shown in FIG. 3.

In one embodiment, a “limonoid glycoside” is a limonoid glucoside. In this embodiment, the glycosylated δ-glycosylated limonoid A-ring carboxylic acid structure further comprises one D-glucose molecule attached via β-glycosidic linkage at C-17 e.g., limonin 17-beta-D-glucopyranoside, nomilin 17-beta-D-glucopyranoside, obacunone 17-beta-D-glucopyranoside, deacetyl nomilinic acid 17-beta-D-glucopyranoside, and the limonoid A-ring D-ring d-glycosylated di-carboxylic acids e.g., nomilinic acid 17-beta-D-glucopyranoside, deacetyl nomilinic acid 17-beta-D-glucopyranoside, and obacunoic acid 17-beta-D-glucopyranoside. Limonoid glucosides are water-soluble triterpenoid compounds that occur naturally in citrus fruit and citrus juice in amounts comparable to vitamin C, and are tasteless in concentrations as high as 600 ppm in orange juice.

“Limonoid glycosides” occur as limonoid glycoside carboxylic acids. Typically, limonoid glycoside carboxylic acids occur in two carboxylic acid forms; mono-carboxylic acids or mono-acids, and di-carboxylic acids, or di-acids.

The term “production scale” or equivalent terms as used herein refer to about one or more gram of final product produced in the method.

The term “aromatic selective” refers to functional groups for chromatographic separations that preferentially select aromatic hydrocarbons. Thus an “aromatic selective” separation is a separation based on hydrophobicity. An “aromatic selective” chromatographic medium will effectively separate mixtures comprising aromatic and aliphatic hydrocarbons by preferential and differential retention of the various aromatic compounds in the mixture. Some exemplary aromatic-selective chromatographic media are aromatic-selective reverse phase chemically bonded silicic acid chromatographic medium, which may include, but is not limited to for example, C-phenyl, C-acyclic and/or cyclic alkyl substituted phenyl, C-oxygenated and/or cyano substituted phenyl, C-oxygenated and/or cyano, and acyclic and/or cyclic alkyl substituted phenyl bonded silica.

The term “isolated” refers to a material that is substantially or essentially free from components which are used to produce the material. For compositions of the invention, the term “isolated” refers to material that is substantially or essentially free from components which normally accompany the material in the mixture used to prepare the composition. “Isolated” and “pure” are used interchangeably. Typically, isolated limonoid compounds have a level of purity that, in exemplary embodiments, is expressed as a range. The lower end of the range of purity for the component is about 60%, about 70% or about 80% and the upper end of the range of purity is about 70%, about 80%, about 90% or more than about 90%. Thus when a limonoid compound is more than about 90% pure, the purities are also preferably expressed as a range. The lower end of the range of purity is about 90%, about 92%, about 94%, about 96% or about 98%. The upper end of the range of purity is about 92%, about 94%, about 96%, about 98% or about 100% purity. Purity is determined by any art-recognized method of analysis (e.g., HPLC, or a similar means).

The term “enhanced” refers to any degree of betterment, augmentation embellishment, beautification, strengthening and/or improvement. For example the phrase “enhanced performance” indicates that performance is improved in one state, by comparison to another.

The term “improved” refers to a more desirable condition than previously existed, or alternatively, improved refers to state wherein a more desirable result is achieved under one set of conditions as compared with another. Improvement is demonstrated by any indicia of success, betterment, progression, or amelioration including any objective or subjective parameter such as abatement, remission, and/or diminishing of symptoms or an improvement in an individual's physical or mental well-being. Improvement can be based on objective or subjective parameters; including the results of a physical examination and/or a psychiatric evaluation.

I. Introduction

Limonoids are highly oxygenated triterpenoid derivatives found in members of the Rutaceae and Meliaceae plant families (see e.g., Herman Z., et al. 1992 supra). Limonoid compounds have been shown to be beneficial for human health. Therefore, the demand for purified limonoid compounds which can be included in the formulations for nutritional supplements, nutraceuticals, therapeutics, pharmaceuticals, cosmetics and the like, has increased dramatically in recent years. Along with increased demand for purified limonoid compounds, a need has arisen for methods by which individual purified limonoid compounds can be manufactured at production scale. Therefore, in one aspect, the present invention provides for the first time techniques useful in the production scale manufacture of limonoid compounds Indeed, one of skill in the art given the following disclosure and the knowledge in the art will find that methods for the production scale isolation and purification of individual limonoid compounds are both readily apparent and accessible.

II. Limonoid Compounds

As noted above, limonoid compounds are highly oxygenated triterpenoid compounds found in tissues from members of the Rutaceae and Meliaceae plant families. For example, members of the Family Rutaceae, genus Citrus comprise limonoids in thier juice, fruit tissues, and seeds.

Typically limonoid compounds are classified as belonging to one of five basic structural categories: (1) neutral aglycones e.g., limonin or nomilin; (2) monocarboxylic aglycones e.g., limonoic acid A-ring lactone; (3) dicarboxylic aglycones e.g., nomilinoic acid; (4) monocarboxylic glycosides e.g., limonin glucoside, and (5) dicarboxylic glycosides e.g., nomilinic acid glucoside (Herman et al., 1992 supra).

A. Limonoid Aglycones

Limonoid aglycones are neutral triterpene compounds that may comprise as many as two six-membered lactone rings, a furan ring substitution at C-17, and typically, though not always comprise a C-14,15 epoxide functionality. The limonoid aglycones typically comprise about 1% of the fresh weight of Citrus seeds. Table 1 lists some exemplary limonoid aglycones.

TABLE 1
Some Exemplary Limonoid Aglycones

| Neutral | | Acidic | |
|---|---|---|---|
| 1. | Limonin | 23. | Deacetylnomilinic acid |
| 2. | Nomilin | 24. | Nomilinic acid |
| 3. | Obacunone | 25. | Isoobacunoic acid |
| 4. | Deacetylnomilin | 26. | Epiisoobacunoic acid |
| 5. | Ichangin | 27. | Isolimonic acid |
| 6. | Deoxylimonin | 28. | Limonoic acid A-ring |
| 7. | Deoxylimonol | | lactone |
| 8. | Limonol | 29. | Deoxylimonic acid |
| 9. | Limonyl acetate | 30. | 17-Dehydrolimonoic acid |
| 10. | 7α-Obacunol | | A-ring |
| 11. | 7α-Obacunyl acetate | | lactone |
| 12. | Ichangensin | 31. | Trans-19-hydroxyobacunoic |
| 13. | Citrusin | | acid |
| 14. | 1-(10-19)Abeo-obacun-9(11)-en- | 32. | Calaminic acid^{a} |
| | 7α-yl acetate | 33. | Retrocalaminic acid^{a} |
| 15. | Calamin^{a} | 34. | Cyclocalaminic acid^{a} |
| 16. | Retrocalamin^{a} | 35. | Isoobacunoic acid |
| 17. | Cyclocalamin^{a} | | diosphenol^{a} |
| 18. | Methyl isoobacunoate diosphenol^{a} | 36. | Obacunoic acid^{a} |
| 19. | Methyl deacetylnomilinate^{a} | 37. | 1-(10-19)Abeo-7α- |
| 20. | 6-Keto-7β-deacetylnomilol^{a} | | acetoxy-10β- |
| 21. | Methyl 6-hydroxy isoobacunoate^{a} | | hydroxy-isoobacunoic acid |
| 22. | Isocyclocalamin^{a} | | |

^{a}Isolated from calamondin seeds

More than 30 citrus limonoid aglycones have been isolated and characterized, mostly from the seeds of Citrus species and species of genera closely related to Citrus.

B. Limonoid Glycosides

Limonoid glycosides are derivatives of A-ring and D-ring ionic ester forms of citrus limonoids. An exemplary limonoid glycoside is shown in FIG. 3. Glycosylation at the δ-hydroxy position of the limonoid D-ring ionic ester or of the limonoid A and D-ring di-ionic ester followed by hydrolysis produces β-glycoside mono and dicarboxylic acids (e.g., limonoid mono and di-β-glycosides).

Typically, glucose is the sugar residue found to be associated with the glycosylation of limonoids. Table 2 lists some exemplary limonoid glucosides (from Herman et al., 1992 supra).

TABLE 2
Some Exemplary Limonoid Glucosides

| Monocarboxylic acids | Dicarboxylic acids |
|---|---|
| 17-β-D-glucopyranosides of: | 17-β-D-glucopyranosides of: |
| 1. Limonin | 5. Nomilinic acid |
| 2. Nomilin | 6. Deacetylnomilinic acid |
| 3. Deacetylnomilin | 7. Obacunoic acid |
| 4. Obacunone | 8. Trans-obacunoic acid |
| | 9. Isoobacunoic acid |
| | 10. Epiisoobacunoic acid |

C. Sources of Limonoid Aglycones and Limonoid Glycosides

Limonoid compounds are obtained from any material comprising limonoid compounds, typically fruits, peels, and seeds of plants from the Families Rutaceae and Meliaceae. For example, citrus fruit tissues and by-products of juice processing such as peels and molasses are ready sources of limonoid glucosides. Citrus seeds also contain high concentrations of both limonoid aglycones and glucosides, and thus are a ready source of limonoid compounds. Indeed, citrus seeds contain the highest concentration of limonoid compounds of any citrus tissue.

Limonoid compound containing material can be in any form, for example whole fresh fruit, whole dried fruit, particulate solid material, a liquefied suspension of solids and/or particulate solid dried extract obtained from Citrus species or species of genera closely related to Citrus; e.g., citrus seeds, processed citrus seed meal or extracts, filtrates or enzymatic digests of citrus seeds or citrus seed meal. In other embodiments the limonoid compound containing material is in the form of water soluble extracts, filtrates or enzymatic digests of limonoid compound containing material e.g., citrus fruit components, citrus juice processing by-products or processed Citrus including peel, pulp, core, seeds, juice, juice processing pulp wash, processed Citrus molasses and citrus fruit sections or obtained from species of genera closely related to Citrus.

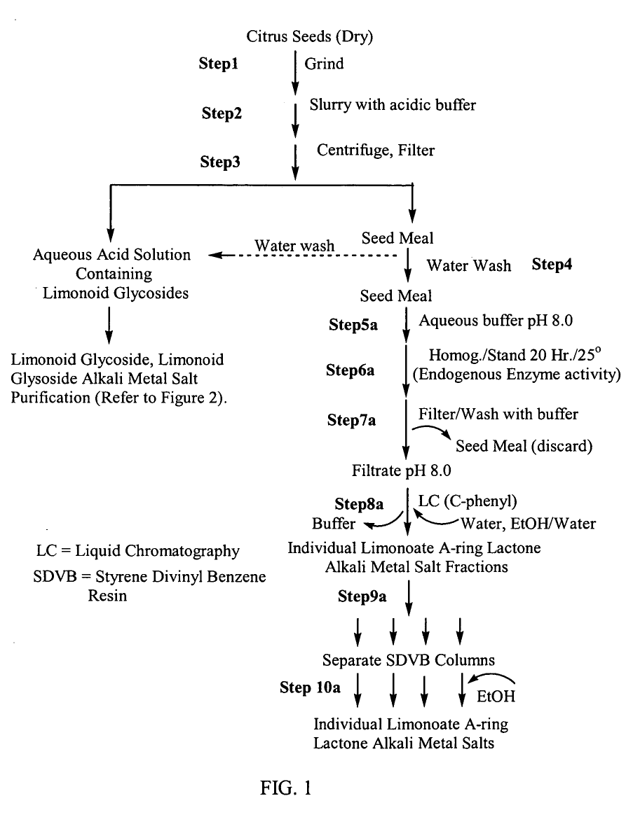

In an exemplary embodiment, the limonoid compound containing material is a solid material such as citrus seed, and the material is treated by for example, by grinding to reduce the particle size of the material. In an exemplary embodiment, the size of the majority of the particles of a ground particulate solid sample has an average particle size about 3 mm and in some embodiments the average particle size is about 1 mm (see e.g., FIG. 1, step 1).

I. Isolating Limonoid Compounds

A. General Methods

In general, limonoid compounds are prepared for isolation from any source containing them by methods well known in the art. Sources of limonoid compounds may be fresh or dried or otherwise preserved, and may be pulped, mashed, ground, chopped, minced, juiced or otherwise manipulated prior to being subject to isolation and purification schemes disclosed herein. Methods of preparing sources of limonoid compounds for isolation and purification are well known in the art (see e.g., Herman et al. 1992 supra).

B. Centrifugation and Filtration

The invention relies on routine techniques of separation such as centrifugation and filtration. Centrifugation is well known in the art (see e.g., Industrial Centrifugation Technology Wallace Woon-Fong Leung ed. McGraw-Hill (1998) and Principles and Techniques of Biochemistry and Molecular Biology Keith Wilson, John eds. Cambridge University Press sixth edition (2005) each of which are incorporated herein by reference in their entirety).

Filtration methods are also well known in the art (see e.g., Filtration in the Biopharmaceutical Industry Meltzer, T. and Jornitz M. W., eds. Marcel Dekker 1998 and Filters and Filtration Handbook, T Christopher Dickenson Elsevier 1997).

C. Chromatography

The invention also utilizes routine techniques in the field of chromatography. Chromatographic methods are well known in the art (see e.g., Preparative and Production Scale Chromatography, Ganetsos, G., and Barker, P. E. eds. 1992 which is incorporated herein by reference). Chromatography is a technique by which the components in a sample, carried by the liquid or gaseous phase, are resolved by sorption-desorption steps on a stationary phase. Thus, in general, chromatography is a separation method that relies on differences in partitioning behavior between a flowing mobile phase and a stationary phase to separate the components in a mixture.

A column (or other support) holds the stationary phase and the mobile phase carries the sample through it. Sample components that partition strongly into the stationary phase spend a greater amount of time in the column and are separated from components that stay predominantly in the mobile phase and pass through the column faster.

As the components elute from the column they can be quantified by a detector and/or collected for further analysis. In some embodiments an analytical instrument maybe combined with a separation method for on-line analysis e.g., liquid chromatography with mass spectrometry (LC-MS).

Limonoid compounds can be separated from each other, and from other components in solution on the basis of size, net surface charge, hydrophobicity, affinity for ligands or any other suitable parameter by which different chemical compounds are distinguished. All of these methods are well known in the art. It will be apparent to one of skill that chromatographic techniques can be performed at any scale and using equipment from many different manufacturers (e.g., Pharmacia Biotech).

1. Ion Exchange Chromatography

As is known in the art, ion exchange chromatography separates molecules based on differences in their overall charge (see e.g., Handbook of Separation Techniques for Chemical Engineers, Philip A Schweitzer ed. McGraw-Hill (1996) and Advances in Chromatography Phyllis R Brown, Eli Gruska eds. Marcel Dekker (1995) each of which is incorporated herein by reference in their entirety). Typically, the molecule of interest has a charge opposite that of the functional group attached to the resin thus permitting the binding of the molecule to the chromatographic medium. For example, molecules which generally have an overall positive charge, will bind well to cation exchangers, which contain negatively charged functional groups. Conversely, molecules which generally have an overall negative charge will bind well to anion exchangers, which contain positively charged functional groups. Because the binding interaction is ionic, binding typically takes place under low ionic conditions. As is known in the art, elution is achieved by increasing the ionic strength to break up the ionic interaction, or by changing the pH of the solution so as to manipulate the charge on the molecule of interest.

Of particular interest, an anion-exchange separation is a process wherein fixed positive charges in one phase, usually solid but occasionally liquid, bind negative molecules in a second phase, usually liquid, contacting the first phase. The bound negative molecules can be separated from electrically neutral or positive molecules in the second phase simply by separation of the two phases. They can be separated from one another by contacting the first phase with fresh liquid of different composition from the original second phase such that the new composition weakens the attraction of more weakly bound anions to the first phase more than it does the attraction of more strongly bound anions to the first phase. Strength of anion attraction to the first phase varies directly with total negative charge of the anion.

A bound anion is “eluted” when a new liquid succeeds in displacing it from the first phase. If the second phase is repeatedly replaced with liquids which progressively interfere more and more strongly with anion binding to the first phase, the process is called a “gradient elution.” If the eluting liquid is changed in composition smoothly over time rather than in successive steps, the gradient elution is “continuous”; otherwise it is “stepwise” elution.

In an exemplary embodiment, the first phase is a solid. This “anion-exchange solid” comprises an electrically neutral “backbone” material which defines its size, shape, porosity, and mechanical properties, and positively charged “functional groups”, preferably attached covalently to the backbone. Typically the backbone materials comprise silica, polysaccharides, and synthetic polyolefins e.g., polystyrene and the polyacrylics. The latter comprise polymers of various substituted acrylic acid amides (“polyacrylamides”) and acrylic acid esters (“polyacrylates”), wherein the acrylic monomer may or may not have alkyl substituents on the 2- or 3-carbon.

Anion exchangers may be “strong” or “weak”. As is known in the art, a strong anion exchanger is one which remains almost fully ionized over a wide pH range, e.g., Triethylaminomethyl (C₂H₄N⁺(C₂H₅)₃), Triethylaminoethyl (C₂H₄N⁺(C₂H5)₂CH₂CH(OH)CH₃), Diethyl-2-hydroxypropylaminoethyl. Thus, a strong anion exchanger is able to bind anions over a wide range of pH. In contrast, a weak anion exchanger ionized over a small pH range e.g., Aminoethyl (C₂H₄N⁺H₃), Diethylaminoethyl (C₂H₄NH(C₂H₅)₂), and therefore is able to bind anions only over a small pH range.

Typically, in anion-exchange separations, the eluting liquid is an aqueous electrolyte; and gradient elution is accomplished by increasing the concentration of a completely dissociated salt dissolved in the water. Increasing the eluting salt concentration in the anion-exchange solvent weakens the binding of anions to the anion-exchange solid.

2. Aromatic Selective Chromatography

Aromatic selective chromatography is a chromatographic method used to separate mixtures of organic compounds based upon aromatic character. Typically the functional groups attached to the solid phase of aromatic selective chromatography media have a high degree of aromatic character, and thus have affinity for organic compounds based upon their degree of aromaticity. Some useful functional groups bonded to the silica based aromatic selective chromatography media include, but are not limited to C-phenyl, C-acyclic and/or cyclic alkyl substituted phenyl, C-oxygenated and/or cyano substituted phenyl, C-oxygenated and/or cyano, and acyclic and/or cyclic alkyl substituted phenyl. Thus in some embodiments useful aromatic selective chromatographic media include, but are not limited to aromatic-selective reverse phase chemically bonded silicic acid chromatographic medium, which may include, but is not limited to for example, C-phenyl, C-acyclic and/or cyclic alkyl substituted phenyl, C-oxygenated and/or cyano substituted phenyl, C-oxygenated and/or cyano, and acyclic and/or cyclic alkyl substituted phenyl bonded silica. see e.g., CRC Handbook of Chromatography: Liquid Chromatography of Polycyclic Aromatic Hydrocarbons, Hamir S., Ph.D CRC Press 1993 which is incorporated herein by reference in its entirety.

III. Purification of Limonoid Aglycones

A. Obtaining a Mixture of Limonoid A-Ring Lactone Acid Salts From Seed Solids

An exemplary method for obtaining a mixture of limonoid A-ring lactone acid salts is shown in FIG. 1 and is described in detail in Example 1. Thus in one exemplary embodiment, the invention comprises methods to obtain a mixture of limonoid A-ring lactone acid salts (FIG. 1, steps 5a-7a).

Seed solids or other suitable staring material comprising limonoid aglycones, is suspended in an aqueous basic buffer solution. In one embodiment the pH of the buffer is in a range between about pH 7.5 to about pH 9.5. In another embodiment the pH of the buffer is about pH 8 (FIG. 1, step 5a). The seed solid suspension is mixed thoroughly e.g., by homogenization in a Waring blender. Typically homogenization is completed in about 5 min.

The homogenized solution is then treated so as to allow the endogenous enzyme limonin D-ring hydrolase (E. C. 3.1.1.36) (Maier et al.,1969) to open the D-ring of the water-insoluble limonoid aglycones thereby forming water-soluble limonoid A-ring lactone limonoates (FIG. 1, step 6a). Typically the homogenized solution is stored at about 25° C. for about 20 hr to affect the ring opening. However storage for shorter or longer periods at higher or lower temperatures is also effective. For example, 25° C. for 19 hr, 18 hr, 17 hr, 16 hr, 15 hr, 14 hr, 13 hr, 12 hr, 11 hr, or 10 hr, or e.g., 20° C. for 19 hr, 18 hr, 17 hr, 16 hr, 15 hr, 14 hr, 13 hr, 12 hr, 11 hr, or 10 hr. The exact time and temperature are not critical, only that there is sufficient time for some of the ring opening reaction to take place.

In an alternative embodiment, limonin D-ring hydrolase is added to the homogenized aqueous suspension of limonoid aglycones to convert the aglycones to water-soluble limonoid A-ring lactone limonoates. Limonin D-ring hydrolase may be added anytime during the storage treatment described above, or may be used in place of the storage treatment described above.

After treatment to effect D-ring opening, homogenate suspension is treated to separate the solid and liquid phases. In one embodiment, solid and liquid phases are separated by centrifugation. In another embodiment, the separated liquid phase is decanted and filtered through diatomaceous earth (FIG. 1, step 7a).

Thus, unlike other methods (see e.g., Miyake, M., et al. (1991) Nippon Nogeikagaku Kaishi 65:987-992) in an exemplary embodiment, an alkaline filtrate is obtained which comprises a mixture of limonoid A-ring lactone acid salts in an open D-ring conformation.

B. Obtaining Individual Pure Limonoid A-Ring Lactone Acid Salts From a Mixture

An exemplary method for obtaining individual pure limonoid A-ring lactone acid salts is shown schematically in FIG. 1 (FIG. 1, steps 8a-10a). One embodiment is also described in Example 1. In one embodiment, the method comprises: applying an aqueous mixture of limonoid A-ring lactone acid salts to an aromatic-selective chromatographic medium and eluting the medium with a suitable polar solvent e.g., ethanol, methanol, isopropanol, acetonitrile, or a combination of these. The aqueous mixture of limonoid A-ring lactone acid salts is obtained by any method known in the art, e.g., the method disclosed in section A, above. Elution of the aromatic-selective chromatographic medium with a polar solvent is conducted so as to differentially elute at least one individual pure limonoid A-ring lactone acid salt from any other limonoid A-ring lactone acid salt (FIG. 1, step 8a).

In an exemplary embodiment, the aromatic-selective chromatographic medium is aromatic-selective reverse phase chemically bonded silicic acid chromatographic medium, which may include, but is not limited to for example, C-phenyl, C-acyclic and/or cyclic alkyl substituted phenyl, C-oxygenated and/or cyano substituted phenyl, C-oxygenated and/or cyano, and acyclic and/or cyclic alkyl substituted phenyl bonded silica.

The one or more individual limonoid A-ring lactone acid salts (denoted as individual limonoate A-ring lactone alkali metal salt fractions in FIG. 1, step 8a) are collected and screened for alkali metal salts of limonoid A-ring lactones as known in the art, such as by liquid chromatography-ultraviolet (LC-UV) or liquid chromatography-mass spectroscopy (LC-MS) (Breksa III, A. P., Manners, G. D. (2004) J. Agric. Food Chem. 52:3772-3775, which is incorporated herein by reference in its entirety).

Fractions determined to contain individual limonoid A-ring lactone acid salts are combined to form a fraction comprising a separated individual limonoid A-ring acid salt. Each combined fraction is applied onto a medium that retains the acid salt (FIG. 1, step 9a). In an exemplary embodiment, such media include, but are not limited to an unmodified polystyrene divinyl benzene copolymer (SVDB), an oxygenated and/or alkylated modified polystyrene divinyl benzene copolymer resin.

Individual pure limonoid A-ring lactone acid salts are eluted from each column with an alcohol solvent e.g., ethanol, methanol (FIG. 1, step 10a). The eluates are collected and concentrated, e.g., on a rotary evaporator (in vacuo) as amorphous solids to obtain individual pure limonoid A-ring lactone acid salts having a purity of about 90%, about 91%, about 92%, about 93%, about 94%, about 95%, about 96%, about 97%, about 98%, about 99% or greater.

III. Purification of Limonoid Glycosides

Limonoid glycosides can be purified for use in a number of applications including, but not limited to ingredients in nutritional supplements, nutraceuticals, pharmaceuticals, cosmetics and the like.

A. Separation of Limonoid Glycoside Monocarboxylic Acids from Limonoid Glycoside Dicarboxylic Acids

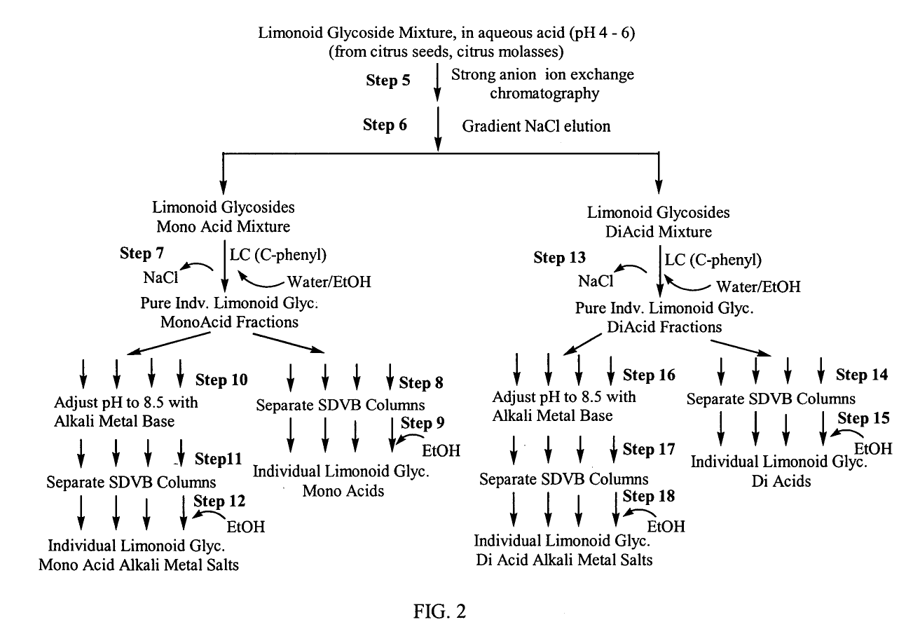

In an exemplary embodiment, shown schematically in FIG. 2, the invention comprises methods for separating limonoid glycoside monocarboxylic acids and limonoid glycoside dicarboxylic acids. In an exemplary embodiment the method comprises: applying the solution at a pH that is in a range of between about pH 4 to about 6.5 to a strong anion ion exchange resin e.g., microporous gel quaternary alkyl, alkylbenzyl substituted ammonia, macroreticular bead quaternary alkyl, alkylbenzyl ammonia and quaternary alkyl, alkylbenzyl ammonia bonded silica gel ion exchange media, and/or Q Sepharose (see e.g., FIG. 2, step 5); eluting the resin with a linear gradient of a suitable solvent e.g., a counter ion that will differentially displace bound limonoid glycoside monocarboxylic acid and limonoid glycoside dicarboxylic acid from the strong anion ion exchange resin, e.g., a linear gradient of sodium chloride (see e.g., FIG. 2, step 6); and collecting the eluted limonoid glycoside monocarboxylic acids separately from the eluted limonoid glycoside dicarboxylic acids.

In one embodiment, the solution comprising limonoid glycoside monocarboxylic acids and limonoid glycoside dicarboxylic acids has a pH that is in a range of between about pH 4 to about 6.5.

B. Separation of Individual Pure Limonoid Glycoside Monocarboxylic Acids from a Mixture of Limonoid Glycoside Monocarboxylic Acids

In an exemplary embodiment, the invention provides a method for separating limonoid glycoside monocarboxylic acids in a mixture comprising a first limonoid glycoside monocarboxylic acid and at least one other limonoid glycoside monocarboxylic acid e.g., FIG. 2, steps 7-9.

The mixture of limonoid glycoside mono-carboxylic acids can be obtained by any method known in the art e.g., as disclosed in section A, above. In an exemplary embodiment, the mixture of limonoid glycoside monocarboxylic acids is applied to an aromatic-selective chromatographic medium e.g., aromatic-selective reverse phase chemically bonded silicic acid chromatographic medium, including, for example, C-phenyl, C-acyclic and/or cyclic alkyl substituted phenyl, C-oxygenated and/or cyano substituted phenyl, C-oxygenated and/or cyano, and acyclic and/or cyclic alkyl substituted phenyl bonded silica; and the medium is eluted with a suitable polar solvent e.g., a solution of aqueous ethanol, methanol, n-propanol, isopropanol, or acetonitrile. Thus, the first limonoid glycoside monocarboxylic acid and the at least one other limonoid glycoside monocarboxylic acids are eluted through the medium at differential rates providing separate fractions comprising the separated first limonoid glycoside monocarboxylic acid and one or more other separated limonoid glycoside monocarboxylic acids respectively.

Separated fractions are then treated to purify the fractions. To purify the fractions, the separated fractions are applied to separate columns containing a medium that retains limonoid glycoside monocarboxylic acid, e.g., SDVB columns such as an unmodified polystyrene divinyl benzene copolymer or an oxygenated and/or alkylated modified polystyrene divinyl benzene copolymer resin (see e.g., FIG. 2, step 8).

Individual limonoid glucosides are eluted from each of columns with a polar solvent e.g., ethanol and/or methanol. One or more of the collected fractions is concentrated to dryness or near dryness, e.g., on a rotary evaporator (in vacuo) (see e.g., FIG. 2, step 9).

In one embodiment, the individual purified limonoid glucosides concentrates are treated to obtain a crystalline or an analytically certified single component amorphous material. In an exemplary embodiment, crystalization of the concentrate is achieved e.g., through fractional crystallization with selected solvents, e.g., EtOH/water, isopropanol/water, to obtain an individual pure crystalline limonoid glycoside monocarboxylic acid, e.g., limonin glucoside The crystallized concentrates have a purity of about 90%, about 91%, about 92%, about 93%, about 94%, about 95%, about 96%, about 97%, about 98%, about 99% or greater.

C. Methods of the Invention to Obtain Individual Pure Limonoid Glycoside Monocarboxylic Acid Salts

In other exemplary embodiments, the invention provides methods to obtain individual pure limonoid glycoside monocarboxylic acid salts see e.g., FIG. 2, steps 7 and 10-12.

In one embodiment, pure individual limonoid glycoside monocarboxylic acids are isolated e.g., according to the method disclosed in section B. above, e.g., FIG. 2, step 7). The isolated pure individual limonoid glycoside monocarboxylic acid solution is treated with a base, e.g., alkali metal base e.g., sodium hydroxide, potassium hydroxide, calcium hydroxide (see e.g., FIG. 2, step 10). The pH of the aqueous base is sufficiently basic to form the acid salt.

The basified soulution of pure individual limonoid glycoside monocarboxylic acids is applied to a column of medium that retains the fraction e.g, an unmodified polystyrene divinyl benzene copolymer or an oxygenated and/or alkylated modified polystyrene divinyl benzene copolymer resin (see e.g., FIG. 2, step 11). As shown in FIG. 2, step 12, individual limonoid glycoside monocarboxylic acid salts are eluted from the column with suitable polar solvent e.g., ethanol and methanol. The eluted fractions comprising the pure individual limonoid glycoside monocarboxylic acid salts are collected.

In one embodiment one or more of the collected fractions comprising individual limonoid glucoside monocarboxylic acid salt are concentrated to dryness or near dryness, e.g., on a rotary evaporator (in vacuo).

In another embodiment, the individual limonoid glucoside monocarboxylic acid salt concentrates produced by drying, are treated to obtain to a crystalline or an analytically certified single component amorphous material. In an exemplary embodiment, crystalization of the concentrate is achieved e.g., through fractional crystallization with selected solvents, e.g., EtOH/water, isopropanol/water, to obtain an individual pure crystalline limonoid glycoside monocarboxylic acid salt, e.g., alkali metal salt. The crystallized concentrates have a purity of about 90%, about 91%, about 92%, about 93%, about 94%, about 95%, about 96%, about 97%, about 98%, about 99% or greater.

D. Methods to Obtain Individual Pure Limonoid Glycoside Dicarboxylic Acids

In an exemplary embodiment, the invention provides a method for separating limonoid glycoside di-carboxylic acids in a mixture comprising a first limonoid glycoside di-carboxylic acid and at least one other limonoid glycoside di-carboxylic acid e.g., FIG. 2, steps 13-15.

The mixture of limonoid glycoside di-carboxylic acids can be obtained by any method known in the art e.g., as disclosed in section A, above. In an exemplary embodiment, the mixture of limonoid glycoside di-carboxylic acids is applied to an aromatic-selective chromatographic medium e.g., aromatic-selective reverse phase chemically bonded silicic acid chromatographic medium, including, for example, C-phenyl, C-acyclic and/or cyclic alkyl substituted phenyl, C-oxygenated and/or cyano substituted phenyl, C-oxygenated and/or cyano, and acyclic and/or cyclic alkyl substituted phenyl bonded silica; and the medium is eluted with a suitable polar solvent e.g., a solution of aqueous ethanol, methanol, n-propanol, isopropanol, or acetonitrile. Thus, the first limonoid glycoside di-carboxylic acid and the at least one other limonoid glycoside di-carboxylic acids are eluted through the medium at differential rates providing separate fractions comprising the separated first limonoid glycoside monocarboxylic acid and one or more other separated limonoid glycoside monocarboxylic acids respectively.

Separated fractions are then treated to purify the fractions. To purify the fractions, the separated fractions are applied to separate columns containing a medium that retains limonoid glycoside monocarboxylic acid, e.g., SDVB columns such as an unmodified polystyrene divinyl benzene copolymer or an oxygenated and/or alkylated modified polystyrene divinyl benzene copolymer resin (see e.g., FIG. 2, step 14).

Individual limonoid glucosides are eluted from each of columns with a polar solvent e.g., ethanol and/or methanol (see e.g., FIG. 2, step 15). In one exemplary embodiment, one or more of the collected fractions is concentrated to dryness or near dryness, e.g., on a rotary evaporator (in vacuo).

In one embodiment, the individual purified limonoid glucoside concentrates are treated to obtain a crystalline or an analytically certified single component amorphous material. In an exemplary embodiment, crystalization of the concentrate is achieved e.g., through fractional crystallization with selected solvents, e.g., EtOH/water, isopropanol/water, to obtain an individual pure crystalline limonoid glycoside monocarboxylic acid, e.g., limonin glucoside. The crystallized concentrates have a purity of about 90%, about 91%, about 92%, about 93%, about 94%, about 95%, about 96%, about 97%, about 98%, about 99% or greater.

E. Methods to Obtain Individual Pure Limonoid Glycoside Dicarboxylic Acid Salts

In other exemplary embodiments, the invention provides methods to obtain individual pure limonoid glycoside di-carboxylic acid salts see e.g., FIG. 2, steps 13, 16-18.

In one embodiment, pure individual limonoid glycoside di-carboxylic acids are isolated e.g., according to the method disclosed in section D. above, e.g., FIG. 2, step 13). The isolated pure individual limonoid glycoside monocarboxylic acid solution is treated with a base, e.g., alkali metal base e.g., sodium hydroxide, potassium hydroxide, calcium hydroxide (see e.g., FIG. 2, step 16). The pH of the aqueous base is sufficiently basic to form the acid salt.

The basified soulution of pure individual limonoid glycoside monocarboxylic acids is applied to a column of medium that retains the fraction e.g., an unmodified polystyrene divinyl benzene copolymer or an oxygenated and/or alkylated modified polystyrene divinyl benzene copolymer resin (see e.g., FIG. 2, step 17). As shown in FIG. 2, step 18, individual limonoid glycoside di-carboxylic acid salts are eluted from the column with suitable polar solvent e.g., ethanol and methanol. The eluted fractions comprising the pure individual limonoid glycoside di-carboxylic acid salts are collected.

In one embodiment one or more of the collected fractions comprising individual limonoid glucoside di-carboxylic acid salt are concentrated to dryness or near dryness, e.g., on a rotary evaporator (in vacuo).

In another embodiment, the individual limonoid glucoside di-carboxylic acid salt concentrates produced by drying, are treated to obtain to a crystalline or an analytically certified single component amorphous material. In an exemplary embodiment, crystalization of the concentrate is achieved e.g., through fractional crystallization with selected solvents, e.g., EtOH/water, isopropanol/water, to obtain an individual pure crystalline limonoid glycoside di-carboxylic acid salt, e.g., limonoid glycoside di-carboxylic acid alkali metal salt. The crystallized concentrates have a purity of about 90%, about 91%, about 92%, about 93%, about 94%, about 95%, about 96%, about 97%, about 98%, about 99% or greater.

IV. Pharmaceutical/Nutraceutical Compositions

Purified limonoid compounds isolated by the methods of the invention can formulated into pharmaceutical compositions and for administration by any means known in the art, e.g., parenterally, topically, orally, or by local administration, such as by aerosol or transdermally. Pharmaceutical/nutraceutical formulations comprising the purified limonoid compounds of the invention are used to provide for prophylactic and/or therapeutic treatments.

The purified limonoid compounds as pharmaceutical formulations can be administered in a variety of unit dosage forms depending upon the condition or disease, the general medical condition of each subject, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Therapeutically effective amounts purified limonoid compounds suitable for practice of the method of the invention will typically range from about 0.5 to about 25 milligrams per kilogram (mg/kg). A person of ordinary skill in the art will be able without undue experimentation, having regard to that skill and this disclosure, to determine a therapeutically effective amount of a particular constitutively purified limonoid compounds for practice of this invention.

Pharmaceutically acceptable carriers are determined in part by the particular composition being administered as well as by the particular method used to administer the composition. Accordingly, there are a wide variety of suitable formulations of pharmaceutical compositions of the present invention (see, e.g., Remington's supra).

For example, compositions may take the form of tablets, pills, capsules, semisolids, powders, sustained release formulations, solutions, suspensions, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, may be found in such standard references Remington's supra. Suitable liquid carriers, especially for injectable solutions, include water, aqueous saline solution, aqueous dextrose solution, and glycols.

Aqueous suspensions of the invention contain purified limonoid compounds in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending purified limonoid compounds in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, (1997) J. Pharmacol. Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent

Purified limonoid compounds pharmaceutical formulations can be prepared according to any method known to the art for the manufacture of pharmaceuticals. Such drugs can contain sweetening agents, flavoring agents, coloring agents and preserving agents. Any purified limonoid compounds formulation can be admixtured with nontoxic pharmaceutically acceptable excipients which are suitable for manufacture.

In another embodiment, the purified limonoid compound formulations of the invention are useful for intravenous (IV) administration. The formulations for administration will commonly comprise a solution of the constitutively phosphorylated osteopontin peptide dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of purified limonoid compounds in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

After a pharmaceutical comprising a purified limonoid compound has been formulated in a acceptable carrier, it can be placed in an appropriate container and labeled for treatment of an indicated condition. For administration of purified limonoid compounds, such labeling would include, e.g., instructions concerning the amount, frequency and method of administration.

### EXAMPLES

### Example 1

The following example illustrates a method for isolation and purification of limonoid glucosides and limonoid aglycones from citrus seeds, and for the preparation of the alkali metal salts of the isolated limonoid compounds.

A. Isolation and Purification of Limonoid Glucosides from Citrus Seeds and Formation of Limonoid Glucoside Alkali Metal Salts

a. Isolation and Purification.

Citrus seeds (10 g) were freeze-dried and ground to pass a 2 mm screen (FIG. 1, Step 1). A portion (0.5 g) of the ground seeds were slurried with a HCl buffer solution (pH 4.0) (30 mL) and agitated (30 min.) (FIG. 1, Step 2). The buffer/seed suspension was centrifuged (1500 G, 20 min.), and the supernatant was decanted and filtered through a bed of diatomaceous earth filter aid (FIG. 1, Step 3). The buffer extracted seed material was re-suspended in water (10 mL), agitated (10 min.) and centrifuged (1500 xg, 15 min). The supernatant was decanted and filtered through diatomaceous earth filter aid and added to the original acid buffer filtrate (FIG. 1, Step 4). The resulting washed seed meal was saved as a source for the isolation of limonoid aglycones (see section B, below).

The acidic buffer solution and the water wash were combined and loaded on to a gravity flow strong anion (quaternary amine) ion exchange column (Q Sepharose, Q-1126, Matrix: Sepharose Fast Flow; Approx. Exclusion Limit: Average MW 4×10⁶; Exchange Capacity: 180-250 μeq/mL gel; Binding Capacity: 120 mg HSA per mL gel; pH Stability: 2-12, Sigma). The solution was drawn down to the column packing bed (FIG. 2, Step 5) and the column was eluted (˜10 mL/min) with a linear gradient of NaCl (0.2 M to 0.4 M, 4 L total solvent volume). Fractions (20 ml) were collected and analyzed for limonoid glucoside content by high-pressure liquid chromatography (HPLC) (C-phenyl, 150 mm×2 mm, 2μ, EtOH/water (15:85)) coupled with ultraviolet detection (LC-UV) at 210 nm or mass spectrometry (LC-MS, electrospray, negative ion detection).

Chromatographic fractions were segregated into two groupings: fractions containing limonoid glucoside monoacids and fractions containing limonoid glucoside di-acids. The fractions in each group were combined in a single fraction (FIG. 2, Step 6).

The combined mono- and di-carboxylic acid fractions are treated similarly to achieve purification (FIG. 2, Steps 7-18). In brief, the combined fractions were applied to a C-phenyl bonded silica chromatographic column. The chromatographic column was eluted with a Water/EtOH (90:1) eluent (FIG. 2, Step 7 and Step 13), and fractions from the chromatographic separation were collected and screened for individual limonoid glucoside content by the HPLC liquid chromatographic system described above. Fractions containing individual pure limonoid glucosides were combined, and each of the combined fractions which represented an individual pure limonoid glucoside was loaded onto separate styrene divinyl benzene resin columns (SDVB) (FIG. 2, Step 8 and Step 14).

Individual limonoid glucosides were eluted from each of the SDVB columns with EtOH and each EtOH solution was concentrated to dryness or near dryness on a rotary evaporator (in vacuo) (FIG. 2, Step 9 and Step 15). The individual limonoid glucoside concentrates were fractionally crystallized with selected solvents (EtOH/water, isopropanol/water) to obtain pure crystalline materials, e.g., limonin glucoside, 95% pure.

b. Formation of Alkali Metal Salt.

Chromatographic fractions containing pure limonoid glucosides obtained as disclosed above (e.g, FIG. 2, Steps 7 and 13), are adjusted to pH 8.5 with and aqueous alkali metal base solution (NaOH) (Step 10 and Step 16) and each aqueous alkaline fraction was applied to an SDVB column (50 mm×20 cm) (FIG. 2, Step 11 and Step 17). Each SDVB column was eluted with water to remove excess alkali and then eluted with EtOH to recover the sodium salt of the limonoid glucoside (FIG. 2, Step 12 and Step 18). The EtOH eluent was evaporated to dryness (rotary evaporator, in vacuo) yielding an amorphous solid, the alkali metal salt of the pure limonoid glucoside.

B. Isolation and Purification of Limonoid Aglycones as A-Ring Lactone Alkali Metal Salts from Citrus Seeds

a. Isolation and Purification.

The water washed seed meal produced above (e.g., in Step 4 of FIG. 1) was suspended in an aqueous NaOH buffer solution pH 8.0 (FIG. 1, Step 5a). The suspension was homogenized in a Waring blender for 5 min. The homogenized solution was removed from the blender, placed in an Erlenmeyer flask and stored at 25° C. (20 hr).

During the storage period the enzyme limonin D-ring hydrolase (E.C. 3.1.1.36) (Maier et al.,1969) opens the D-ring of the limonoid aglycones to form the water-soluble limonoid A-ring lactone limonoates (FIG. 1, Step 6a).

Following storage, the suspension was centrifuged and the supernate was decanted and filtered through a celite bed (FIG. 1, Step 7a). The alkaline filtrate was loaded on to a C-phenyl bonded silica chromatographic column.

The chromatographic column was eluted with a Water/EtOH (90:1) eluent (FIG. 1, Step 8a). Fractions are collected and screened for sodium salts of limonoid A-ring lactones by LC-UV or LC-MS.

b. Formation of A-ring Lactone Acid Salts.

Fractions determined to contain individual pure limonoid A-ring lactone acid salts were combined and loaded onto separate styrene divinyl benzene resin columns (SDVB) (FIG. 1, Step 9a). Individual pure limonoid A-ring lactone acid salts were eluted from each SDVB column with EtOH (FIG. 1, Step 10a), and the EtOH solutions were collected and concentrated on a rotary evaporator (in vacuo) as amorphous solids.

### Example 2

The following example illustrates isolation and purification of limonoid glucosides from citrus molasses and the formation of limonoid glucoside alkali metal salts.

A. Isolation and Purification of Limonoid Glucosides from Citrus Molasses

The reclamation of a mixture of limonoid glucosides from crude citrus molasses has been previously described (see e.g., U.S. Pat. No. 5,734,046 to (Ifuku et al., 1998, Schoch et al.,2002) J. Food Sci. 67:3159-3163). The isolation of pure individual limonoid glucosides from this mixture and the formation of alkali metal salts of the pure limonoid glucosides is disclosed in FIG. 2 and described above in Example 1.

After dissolving the mixture of limonoid glucosides in an aqueous acid solution (HCl, pH 6), the procedure is a reiteration of Steps 5-18 of the isolation and purification procedure for limonoid glucosides obtained from citrus seeds described above.

### Example 3

### Ion Exchange as Applied to a Limonid Glucoside Mixture

A. Experimental Description

A strong anion exchange resin (Mitsubishi FP-DA-13, 100 mL) was packed in a glass chromatographic column (24 mm×200 mm) to achieve a bed dimension of 24 mm×140 mm. The resin bed was sequentially eluted with water (200 mL), 1.0N aqueous sodium hydroxide (NaOH) (200 mL), water (200 mL), 1.0N aqueous phosphoric acid (H₃PO₄) (200 mL), and water (300 mL). An aqueous mixture of limonoid glucosides previously obtained from citrus molasses according to a published method (Schoch, T. K., Manners, G. D., Hasegawa, S., J. Food. Sci. 77, 3159, 2002 which is incorporated herein by reference in its entirety) containing the mono-acidic limonoid glucosides limonin glucoside, nomilin glucoside, obacunone glucoside and deacetyl nomilin glucoside and the di-acidic limonoid glucosides deacetyl nomilininic acid glucoside, nomilinic acid glucoside and obacunoic acid glucoside was prepared (0.5 g in 100 ml). The limonoid glucoside mixture was applied to the strong ion exchange resin (4 mL/min). The chromatographic column was eluted (4 mL/min) with water (500 mL) followed by 0.05 M potassium hydrogen phosphate (KH₂PO₄) (600 mL). Fractions (20 mL) were collected and the aqueous eluent fraction were directly qualitatively analyzed for limonoid glucoside content by liquid chromatography-mass spectrometry (LC-MS) (direct infusion) to locate and identify limonoid glucosides. Aliquots (1 mL) of the potassium hydrogen phosphate eluent fractions were applied to previously prepared styrene divinyl benzene solid phase extraction (SPE) modules (100 mg, Isolute 101, International Sorbent Technology). The SPE modules were washed with water (3 mL), run to dryness and eluted with methanol (3 mL). The methanol solution of the di-acid fractions were qualitatively analyzed in the same manner as the mono-acidic limonoid glucosides.

Fractions found to contain mono-acidic limonoid glucosides were combined and applied to a styrene divinyl benzene resin (Mitsubishi SP-70) column (24 mm×100 mm) that had been previously prepared with methanol (MeOH) (100 mL) and water (300 mL). The aqueous mixture of mono-acidic limonoid glucosides was applied to the styrene divinyl benzene resin column (4 mL/min). After addition of the mixture, the resin was eluted with water (100 mL) followed by methanol (MeOH). The methanol eluent was concentrated to dryness (in vacuo) and the total weight of mono-acidic limonin glucosides was determined. Fractions containing di-acidic limonin glucosides were identified, combined and chromatographically reclaimed in the same manner as the mono-acidic limonoid glucosides.

B. Results

The qualitative LC-MS detection of limonoid glucosides revealed that the mono-acidic limonoid glucosides (limonin glucoside, nomilin glucoside, obacunone glucoside and deacetyl nomilin glucoside) were selectively eluted in the water fraction (450 mL). Qualitative LC-MS analysis of the potassium hydrogen phosphate fractions revealed that the di-acidic limonoid glucosides (deacetyl nomilinic acid glucoside, nomilinic acid glucoside and obacunoic acid glucoside) were selectively eluted in the potassium hydrogen phosphate fractions. Gravimetric analysis of the recovered mono-acidic limonoid glucosides was found to be 54% (0.27 g) of the original limonoid glucoside mixture and the recovered di-acid fraction was found to be 39% (0.19 g) of the original mixture.

### Example 4

### C-Phenyl Separation of Limonoid Glucosides

A. Separation of Limonoid Glucosides by Aromatic Selective Reverse Phase Chromatography

Citrus molasses (250 mL) containing a mixture of limonoid glucosides was diluted to 1 L with water and clarified utilizing a strong cation exchange resin as described in a published procedure (Schoch, T. K., Manners, G. D., Hasegawa, S. J. Food Sci. 67, 3159, 2002 which is incorporated herein by reference in its entirety). An aliquot (5 mL) of the water eluent from the strong cation exchange column was applied to a C-Phenyl solid phase extraction (SPE) module (Strata Phenyl, 500 mg bed, 3 mL, Phenomenex) that had previously be washed with ethanol (EtOH) and water sequentially. The molasses derived aliquot was sequentially eluted with six aliquots (2 mL) of water, 90% aq. EtOH, 80% aq. EtOH, and 100% EtOH. Each of the eluents were subjected to liquid chromatography-mass spectrometry (C-18 BDS, MeOH/4.0 mm Formic Acid). The chromatographic analysis (notebook PCE-1, pp62-63) revealed that the C-phenyl SPE module could selectively retain the aromatic based coloring material in citrus molasses and selectively chromatographically separate the limonoid glucosides

B. Separation of Mono-acidic and Di-acidic Limonoid Glucosides by Aromatic Selective Reverse Phase Chromatography

Aliquots (1 mL) were taken from mono-acidic and di-acidic fractions obtained from the strong anion exchange chromatography of a mixture of limonoid glucosides isolated from citrus molasses according to a previously published method (Schoch, T. K., Manners, G. D., Hasegawa, S. J. Food Sci. 67, 3159, 2002 which is incorporated herein by reference in its entirety). The mono-acid fraction had been determined to contain limonin glucoside, nomilin glucoside, obacunone glucoside and deacetyl nomilin glucoside by infusion liquid chromatography-mass spectrometry (LC-MS). The di-acid fraction had been determined to contain nomilinic acid glucoside, deacetyl nomilinic acid glucoside, and obacunoic acid glucoside by the same LC-MS method. A portion (10 μL) of each aliquot was subjected to LC-MS chromatography utilizing an aromatic selective C-phenyl reverse

phase chromatographic column (3 mm×150 mm, 5μ, Phenosphere Next Phenyl) and EtOH/H₂O (10/90, isocratic) as the solvent system.

The chromatographic results showed that the mono-acid limonoid glucosides are individually separated in the order limonin glucoside, deacetyl nomilin glucoside, nomilin glucoside and obacunone glucoside. In the case of the di-acids, the limonoid glucosides are individually separated in the order deacetyl nomilinic acid glucoside, obacunoic acid glucoside and nomilinic acid glucoside.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A method for separating limonoid glycoside mono-carboxylic acids from limonoid glycoside di-carboxylic acids in an aqueous solution, the method comprising:
(a) acidifying the aqueous solution to achieve a pH of less than 6.5;
(b) applying the acidified aqueous solution from step (a) to a strong anion ion exchange resin;
(c) eluting the strong anion ion exchange resin with a linear gradient of an ionic solvent; and
(d) collecting eluate fractions that comprise separated limonoid glycoside mono-carboxylic acids and separated limonoid glycoside di-carboxylic acids.

2. The method of claim 1, wherein the strong anion exchange resin comprises quaternary ammonium groups.

3. The method of claim 1, wherein the ionic solvent comprises an aqueous solution of counter ion.

4. The method of claim 3, wherein the aqueous solution of counter ion is a solution of NaCl.

5. The method of claim 1, further comprising steps for separating an individual species of limonoid glycoside mono-carboxylic acid from other species of limonoid glycoside mono-carboxylic acid in an aqueous solution, the steps comprising:
(e) applying the collected eluate fractions comprising limonoid glycoside mono-carboxylic acids from step (d) to an aromatic-selective chromatographic medium;
(f) eluting the aromatic-selective chromatographic medium with a polar solvent; and
(g) collecting eluate fractions comprising the separated individual species of limonoid glycoside mono-carboxylic acid,
thereby separating the individual species of limonoid glycoside mono-carboxylic acid from the other species of limonoid glycoside mono-carboxylic acid in an aqueous solution.

6. The method of claim 1, further comprising steps for separating an individual species of limonoid glycoside di-carboxylic acid from other species of limonoid glycoside di-carboxylic acids in an aqueous solution, the steps comprising:
(e) applying the collected eluate fractions comprising limonoid glycoside di-carboxylic acids from step (d) to an aromatic-selective chromatographic medium;
(f) eluting the aromatic-selective chromatographic medium with a polar solvent; and
(g) collecting eluate fractions comprising the separated individual species of limonoid glycoside di-carboxylic acid,
thereby separating an individual species of limonoid glycoside di-carboxylic acid from other species of limonoid glycoside di-carboxylic acid in an aqueous solution.

7. The method of claim 5, wherein the aromatic-selective chromatographic medium is a phenyl-bonded silica medium.

8. The method of claim 5, wherein the polar solvent is a member selected from the group consisting of aqueous ethanol, aqueous methanol, aqueous n-propanol, and aqueous acetonitrile, or a combination of such members.

9. The method of claim 8, wherein the polar solvent is aqueous ethanol solution.

10. The method of claim 9, wherein the aqueous ethanol solution comprises water and ethanol in a water to ethanol ration about 90:1.

11. The method of claim 5, further comprising:
(h) combining eluate fractions comprising the same isolated individual species of limonoid glycoside mono-carboxylic acid;
(i) applying the combined eluate fractions from step (h) to a styrene divinylbenzene resin;
(j) eluting the styrene divinylbenzene resin with a polar solvent;
(k) collecting eluate fractions comprising the individual species of limonoid glycoside carboxylic acid;
(i) evaporating the polar solvent present in the collected fractions to produce a limonoid glycoside carboxylic acid concentrate; and
(j) fractionally crystallizing the limonoid glycoside carboxylic acid concentrate,
thereby producing a pure crystalline form of an individual species of limonoid glycoside carboxylic acid.

12. The method of claim 11, wherein the styrene divinylbenzene resin is eluted with a polar solvent that is a member selected from the group consisting of aqueous ethanol, aqueous methanol, aqueous n-propanol, and aqueous acetonitrile, or a combination of such members.

13. The method of claim 11, wherein the limonoid glycoside carboxylic acid concentrate is fractionally crystallized using a solution comprising a polar solvent and water.

14. The method of claim 11, wherein the polar solvent is a member selected from the group consisting of aqueous ethanol, aqueous methanol, aqueous n-propanol, and aqueous acetonitrile, or a combination of such members.

15. The method of claim 5, further comprising:
(h) combining eluate fractions comprising the same individual species of limonoid glycoside mono-carboxylic acid;
(i) adding an aqueous alkali metal base to the combined fractions to make the combined fractions alkaline;
(j) applying the alkaline eluate fractions from step (i) to a styrene divinylbenzene resin;
(k) washing the styrene divinylbenzene resin with water to remove excess alkali;
(l) eluting the styrene divinylbenzene resin with a polar solvent;
(m) collecting eluate fractions comprising the individual species of limonoid glycoside mono-carboxylic acid; and
(n) evaporating the polar solvent present in the collected fractions to yield an amorphous solid comprising the alkali metal salt of the individual species of limonoid glycoside mono-carboxylic acid.

16. The method of claim 20 wherein, the aqueous alkali metal base is aqueous NaOH.

17. An alkali metal salt of an isolated limonoid glycoside carboxylic acid made according to the method of claim 15.

18. The method of claim 6, further comprising:
(h) combining eluate fractions comprising the same individual species of limonoid glycoside di-carboxylic acid;
(i) adding an aqueous alkali metal base to the combined fractions to make the combined fractions alkaline;
(j) applying the alkaline eluate fractions from step (i) to a styrene divinylbenzene resin;
(k) washing the styrene divinylbenzene resin with water to remove excess alkali;
(l) eluting the styrene divinylbenzene resin with a polar solvent;
(m) collecting eluate fractions comprising the individual species of limonoid glycoside di-carboxylic acid; and
(n) evaporating the polar solvent present in the collected fractions to yield an amorphous solid comprising the alkali metal salt of the individual species of limonoid glycoside di-carboxylic acid.

19. The method of claim 18, wherein the aqueous alkali metal base is aqueous NaOH.

20. An alkali metal salt of an isolated limonoid glycoside carboxylic acid made according to the method of claim 18.

21. A method of isolating limonoid aglycones from a composition comprising seed solids, the method comprising:
(a) suspending the composition comprising seed solids in a solution of aqueous alkali metal base to form a seed solid suspension;
(b) homogenizing the seed solid suspension from step (a) to form a homogenate;
(c) storing the homogenate from step (b) for a time period of at least 10 hours to form limonoid A-ring lactone limonates from limonoid aglycones;
(d) processing the homogenate to form a solid phase and an alkaline aqueous phase;
(e) collecting the alkaline aqueous phase;
(f) applying the alkaline aqueous phase from step (e) onto a phenyl bonded silica matrix;
(g) eluting the phenyl-bonded silica matrix with a polar solvent; and
collecting eluate fractions comprising limonoid A-ring lactone limonates, thereby isolating limonoid aglycones in the form of limonoid A-ring lactone limonates.

22. The method of claim 21, further comprising the step of:
(i) prior to or during step (c), adding an amount of limonin D-ring hydrolase sufficient to facilitate formation limonoid A-ring lactone limonates from limonoid aglycones.

23. The method of claim 21, further comprising the step of:
(i) prior to or during step (f), filtering the alkaline aqueous phase.

24. The method of claim 21, further comprising:
(h) combining collected fractions comprising limonoid A-ring lactone limonates;
(i) loading the collected eluate fractions from step (j) onto a styrene divinylbenzene resin;
(j) eluting the styrene divinylbenzene resin with a polar solvent;
(k) collecting eluate fractions comprising limonoid A-ring lactones; and
(l) evaporating the polar solvent from the collected fractions to yield an amorphous solid comprising the alkali metal salts of limonoid A-ring lactone limonates, thereby isolating limonoid aglycones.

25. The method of claim 24, wherein the composition comprising seed solids comprises seed solids that are members selected from the group consisting of whole seed, seed extract, seed filtrate, enzymatic digest of seed, seed meal, seed meal extract, seed meal filtrate, and enzymatic digest of seed meal, or a combination of such members.

26. A pharmaceutical or nutraceutical composition comprising an individual pure limonoid compound.
